(19) United States
(12) Patent Application Publication
(10) Pub. No.: US 2012/0035666 A1
(43) Pub. Date: Feb. 9, 2012

(54) Reduced Bone Fracture Fixation Device

(75) Inventors: Amy L. Ladd, Stanford, CA (US); Arnold-Peter C. Weiss, Barrington, RI (US); Gary B. Hulme, San Jose, CA (US); Matthew Thompson, Corte Madera, CA (US); Jeffrey J. Christian, Morgan Hill, CA (US)

(73) Assignee: Osteospring Medical, Inc.

(21) Appl. No.: 13/211,122

(22) Filed: Aug. 16, 2011

Related U.S. Application Data
(63) Continuation-in-part of application No. PCT/US2010/024753, filed on Feb. 19, 2010.
(60) Provisional application No. 61/494,294, filed on Jun. 7, 2011.
(60) Provisional application No. 61/382,331, filed on Sep. 13, 2010.
(60) Provisional application No. 61/375,700, filed on Aug. 20, 2010.
(60) Provisional application No. 61/208,279, filed on Feb. 21, 2009.

(51) Int. Cl. A61B 17/84; A61B 17/56

(52) U.S. Cl. 606/301; 606/300; 606/86 R

(57) ABSTRACT

Reduced bone fracture fixation devices and methods for using the same are provided. Aspects of the reduced bone fracture fixation devices include a body dimensioned to be positioned in a reduced bone fracture, wherein the body includes at least one tension element configured to exert a force on bone of the reduced bone fracture sufficient to maintain reduction of the reduced bone fracture. Aspects of the invention further include kits and methods of using and manufacturing the bone fracture fixation devices. The devices, kits and methods of the invention find use in a variety of applications, such as in applications in which it is desired to repair a reduced bone fracture.

### CROSS-REFERENCE TO RELATED APPLICATIONS

Pursuant to 35 U.S.C. §119 (e), this application claims priority to U.S. Provisional Application Ser. No. 61/494,294 filed on Jun. 7, 2011; U.S. Provisional Patent Application Ser. No. 61/382,331, filed on Sep. 13, 2010; U.S. Provisional Patent Application Ser. No. 61/375,700, filed on Aug. 20, 2010; the disclosures of which are herein incorporated by reference.

This application is also a continuation in part application of PCT application Serial No. PCT/US10/24753 filed on Feb. 19, 2010; which application claims priority to U.S. Provisional Patent Application Ser. No. 61/208,279, filed on Feb. 21, 2009; the disclosures of which applications are herein incorporated by reference.

### INTRODUCTION

Bone or fracture voids may occur in many different types of bones in many different ways. For example, an unstable distal radius fracture is common especially in the endemic osteoporotic populations of North America, Europe, Asia, and Australia. This type of low energy fracture may be sustained by a fall on an outstretched hand. The classic, osteopenic osteoporotic fragility fracture is extra-articular or includes a simple intra-articular component, i.e., the fracture is primarily outside of a joint or may include a simple component within the joint. The fracture may result in dorsal comminution, loss of radial height, loss of volar tilt, radial shift, and shortening. In this regard, dorsal comminution refers to pulverization of the bone in the wrist in the direction of the back of the hand, loss of radial height refers to loss of height in the wrist on the side near the thumb, loss of volar tilt refers to loss of tilt in the wrist in the direction of the palm of the hand, and radial shift refers to shift of the wrist towards the side of the thumb. In addition, poor bone mineral quality and the degree of comminution, especially with proximal extension on the radial column, may render this fracture unstable, such that closed treatment alone may be insufficient. Further, the forces experienced by the wrist during daily activities are primarily compression, e.g., digital motion, and shear/torsion, e.g., forearm rotation. Fracture, e.g., catastrophic collapse, occurs typically in tension, thereby creating a relatively transverse fracture across the metaphysis, the metaphysis being the part of a bone between the shaft of the bone, i.e. diaphysis, and the end of the bone, i.e., epiphysis. The position of the wrist, the forces applied, and the bone quality may determine other components of the fracture, such as, for example, extension into the joint, extension into the diaphysis, and more oblique components from torsional forces.

Reduction, i.e., architectural restoration, of a simple but unstable fracture may be obtained through a variety of means. Although there has been a historical preference for non-operative treatment, more invasive treatments intended to restore cortical, i.e. external or surface, integrity have historically included pins and plaster techniques, external fixation, and cross metaphyseal pinning. Later treatment techniques have included dorsal plating systems that address the radial column, and volar plate fixation. Examples of dorsal plating systems include, e.g., Forte Zimmer low profile plate or Synthes pi plate. The more rigid construct required for volar fixation, given its application on the compression side of the radius, has been purportedly outweighed by soft tissue coverage of the volar plate not afforded by dorsal plating systems.

Although plating systems may address cortical reconstitution, they do not address metaphyseal voids that are formed when osteopenic/osteoporotic bone collapses. Further, rigid volar plates may not adequately overcome the loss of cancellous bone in the metaphysis when significant comminution and severe loss of bony architecture has occurred. To fill these metaphyseal voids, patients' autograft bone, banked allograft bone, and/or synthetic fillers, e.g., calcium phosphate or calcium sulfate, may be used. Moreover, although PMMA (polymethylmethacrylate) cement has historically been used as a void filler, this material is rarely used in radius fractures since biologic and biologically active alternatives are preferred.

Plating systems and volar plate fixation may be more substantial and invasive than a patient's bone or fracture void and co-morbidities may warrant. While such fractures may frequently be reduced (architectural reconstitution) by closed manipulation and successfully casted, follow-up examination in the casting period over the next few weeks often shows that fragility fractures experience loss of reduction with resulting deformity. The typical patient with a fragility fracture is elderly and has co-morbid health conditions, which underscores the importance of minimizing risk at the same time as improving treatment methods. In these patients, it is the maintenance of the fracture reduction that is the challenge rather than obtaining a satisfactory reduction in the first place. Aggressive open fracture treatment is best avoided if it is not necessary to obtain reduction.

### SUMMARY

Reduced bone fracture fixation devices and methods for using the same are provided. Aspects of the reduced bone fracture fixation devices include a body dimensioned to be positioned in a reduced bone fracture, wherein the body includes at least one tension element configured to exert a force on bone of the reduced bone fracture sufficient to maintain reduction of the reduced bone fracture. Aspects of the invention further include kits and methods of using and manufacturing the bone fracture fixation devices. The devices, kits and methods of the invention find use in a variety of applications, such as in applications in which it is desired to repair a reduced bone fracture.

### BRIEF DESCRIPTION OF THE FIGURES

FIG. 1 is a schematic antero-posterior view of a distal radial bone fracture.

FIG. 2 is a schematic side view of a distal radial bone fracture.

FIGS. 3A-B are schematic perspective views of a spring screw embodiment of a reduced bone fracture fixation device.

FIG. 4 is a schematic view of an embodiment of a reduced bone fracture fixation device.

FIG. 5 is a schematic three-dimensional view of an embodiment of a reduced bone fracture fixation device in a fracture site.

FIG. 6 is a schematic view of an embodiment of a reduced bone fracture fixation device.

FIGS. 7A-D are schematic perspective views of an embodiment of a continuous wire embodiment of a reduced bone fracture fixation device.

FIGS. 8A-C are schematic perspective views of a pivot coil embodiment of a reduced bone fracture fixation device.

FIGS. 9A-B are schematic perspective views of an embodiment of a reduced bone fracture fixation device.

FIGS. 10A-C are schematic views of an embodiment of a reduced bone fracture fixation device.

FIGS. 11A-B are schematic views of an embodiment of a reduced bone fracture fixation device in the un-deployed and deployed configurations.

FIG. 12 is a schematic view of an embodiment of a reduced bone fracture fixation device.

FIG. 13 is a schematic view of an embodiment of a reduced bone fracture fixation device.

FIGS. 14A-D are schematic perspective views of various embodiments of a reduced bone fracture fixation device derived from one or more triangular planar elements.

FIG. 15 is a partially formed embodiment of reduced bone fracture fixation device.

FIGS. 16A-B are schematic perspective views of an embodiment of a method of making a reduced bone fracture fixation device.

FIGS. 17A-B are schematic perspective views of an embodiment of a method of making a reduced bone fracture fixation device.

FIGS. 18A-C are schematic perspective views of an embodiment of a reduced bone fracture fixation device.

FIGS. 19A-K are schematic perspective views of a wire embodiment of a reduced bone fracture fixation device.

### DETAILED DESCRIPTION

Reduced bone fracture fixation devices and methods for using the same are provided. Aspects of the reduced bone fracture fixation devices include a body dimensioned to be positioned in a reduced bone fracture, wherein the body includes at least one tension element configured to exert a force on bone of the reduced bone fracture sufficient to maintain reduction of the reduced bone fracture. Aspects of the invention further include kits and methods of using and manufacturing the bone fracture fixation devices. The devices, kits and methods of the invention find use in a variety of applications, such as in applications in which it is desired to repair a reduced bone fracture.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

In further describing various aspects of the invention in greater detail, embodiments of devices of the invention are reviewed first, followed by descriptions of embodiments of the methods and manufacture and use of the devices, as well as kits that include the devices.

### Devices

Reduced bone fracture fixation devices are devices that are configured to maintain reduction of a reduced a bone fracture. In some instances, the devices can be used to reduce a bone fracture. By “reduced bone fracture” is meant a bone fracture wherein the fractured pieces of bone have been restored to their normal or nearly normal anatomic alignment. The goal of fracture treatment is to maintain the bone in a reduced position, i.e., properly aligned, while the bone heals. Accordingly, a reduced bone fracture is a fracture in which the fractured pieces of bone have been restored to the substantially normal, if not normal, anatomic alignment. As is known in the art, a fracture may be reduced by a variety of different reduction protocols. In some instances, the subject devices can be used to reduce or partially reduce a bone fracture. In some instances, the subject devices can be used to both reduce and maintain reduction of a bone fracture. Reduced bone fractures with which devices of invention find use are fractures that have been reduced using any convenient protocol.

As indicated above, the subject devices are configured to exert a force on the surfaces of a reduced bone fracture to maintain the bone in a reduced (i.e., anatomically or nearly anatomically aligned) position. The subject devices can exert forces that include but are not limited to: distraction forces (i.e., forces in which pieces of bone are forced in opposite directions), torsional forces (to prevent a piece of fractured bone from rotating), expansion forces (to resist compression), retention forces (to prevent a bone fragment from migrating), etc.

The reduced bone fracture fixation devices of the subject invention have a body dimensioned to be positioned in a reduced bone fracture. By “dimensioned to be positioned in a reduced bone fracture” is meant that the body of the device can be any size suitable for positioning in the reduced fracture site of interest, which can include, but is not limited to, the distal radius, proximal humerus, proximal tibia, calcaneus, vertebral body, hip, etc. The dimensions of the subject devices can vary according to the size of the bone at the reduced fracture site of interest, the extent or size of the fracture, the size of the subject (e.g., child or adult), etc. The subject devices can be positioned so that they are located entirely within the fracture site; i.e., no portion of the device extends outside of the bone, which minimizes irritation of the surrounding soft tissues. In certain embodiments, the devices have a longest dimension ranging from 3 mm to 6 cm, such as 4 mm to 5 cm, and including 8 mm to 4 cm. In certain embodiments, when fully deployed, the device will occupy a volume ranging from 27 mm³ to 10 cm³, such as 50 mm³ to 8 cm³ and including 1 cm³ to 6 cm³.

The subject devices include at least one tension element. By “tension element” is meant the portion or portions of the body of a reduced fracture fixation device which is configured to exert a force on bone. The force exerted by the one or more tension elements on bone of a reduced fracture is sufficient to maintain reduction of the reduced bone fracture. In some embodiments, the tension element can include the entire body of the device. The amount of force exerted by the one or more tension elements on the surfaces of the bone at a reduced bone fracture site is sufficient to maintain reduction of the reduced bone fracture. When deployed, the one or more tension elements can exert force on a single bone portion of the reduced bone fracture or two or more different bone portions, depending on a number of factors, such as the particular device configuration, the anatomical structure of the reduced fracture, etc.

The amount and direction of the force exerted by the reduced bone fracture fixation devices of the invention can be determined by a number of factors, including variations in the configuration of the device, differences in the material used to construct the subject devices, differences in the dimensions or thickness of the subject device, etc. In some instances, the force exerted can range, for example, from 2 lb_{f} to 60 lb_{f}, such as from 5 lb_{f} to 40 lb_{f}, or 10 lbs. to 20 lbs. of pound-force (lb_{f}).

The subject devices can have different configurations, which can vary depending on the materials and methods used for constructing the device. In some configurations, the devices are configured to undergo a conformational change from a pre-deployed to a deployed state, where in the deployed state the radial width (i.e., a dimension transecting the longitudinal axis at a right angle) a one of the device is longer than the radial width at the other end of the device, e.g., by a factor of 2-fold or more, such as 5-fold or more, including 10-fold or more. The transition from the pre-deployed to a deployed state may achieved using any convenient protocol, e.g., by removal of a constraining element during implantation of the device, etc. Devices of these configurations are therefore distinguished from those devices disclosed in U.S. Published Patent Application 20090143781. In some configurations, the devices are not “caged” devices, i.e., devices that include a first component present inside of a second cage component, such as those devices described in U.S. Published Patent Application 20090182336. Configurations of the subject devices can include, but are not limited to: a conical screw configuration, configurations that can be constructed of wire or a planar element which can include a biased hinge or spring configuration, a looped or coiled configuration, a leaf spring configuration, a ratcheted configuration, etc. In some instances, the devices can be formed by a combination of any of the above configurations, such as a conical screw configuration combined with a wire element, or a wire embodiment combined with one or more planar embodiments, or a conical screw embodiment combined with both a wire embodiment and a planar embodiment, etc. Embodiments of the subject devices which include these various configurations are discussed further below.

The devices can be manufactured using any convenient protocol. For example, the subject devices can be produced by molding, stamping, or machining a piece of metal; by bending or mechanical manipulation of wire; by folding or mechanical manipulation of a planar element, such as a ribbon, etc.

As mentioned above, devices of the invention are reduced fracture fixation devices. The devices may be configured for use with a wide variety of different types of reduced fractures. Different types of reduced fractures of interest include, but are not limited to: fractures of the radius, ulna, humerus, femur, tibia, fibula, clavicle, scapula, spine, vertebral body, ribs, pelvis, carpal bones, tarsal bones, metacarpals, metatarsals, etc., and the like.

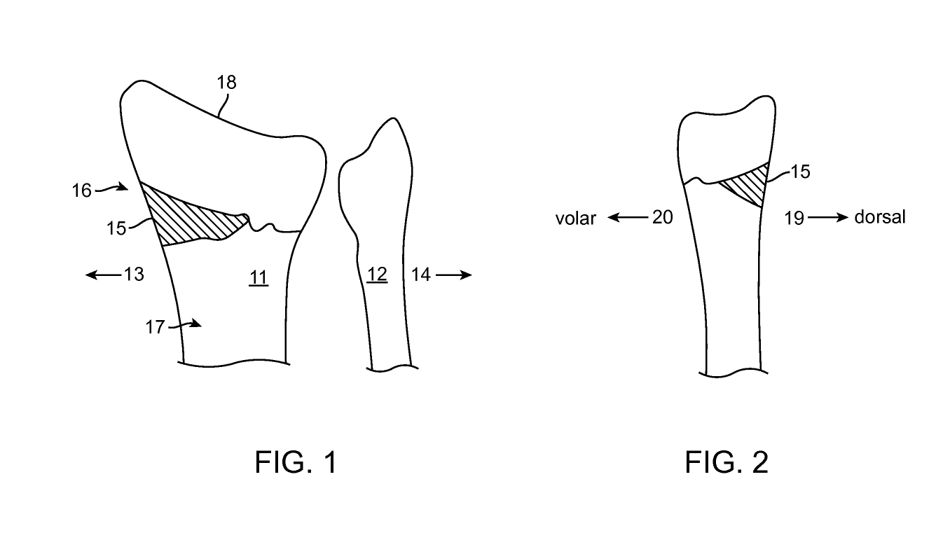

In some embodiments, the reduced fracture is a reduced distal radius fracture. FIG. 1 is a schematic anteroposterior view of a wrist, illustrating a distal radius fracture 15 in the radius 11. Also shown is ulna 12. The distal radial bone fracture 15 is situated on the radial side 13 of the radius 11 opposite the ulnar side 14. The distal radial bone fracture 15 is located in the metaphysis 16 of the radius 11, between the diaphysis 17 and the epiphysis 18 of the radius 11. The bone fracture 15 as shown has created a metaphyseal void (shaded) on the radial side 13 of the radius 11.

FIG. 2 illustrates a schematic side view of a distal radial bone fracture 15, viewed from the radial side of the wrist. FIG. 2 illustrates only the radius 11, since in this view the ulna 12 is substantially hidden behind the radius 11. The side view shows that the bone fracture 15 is located predominantly on the dorsal aspect 19 of the radius 11, opposite the volar side 20. The bone fracture as shown has created a metaphyseal void (shaded) on the dorsal aspect 19 of the radius 11.

The bone fracture 15 illustrated in FIGS. 1 and 2 is an example of an unstable, distal radius fracture that is extraarticular, i.e., the fracture is located outside of a joint. The fracture 15 as shown may result in dorsal comminution, i.e., pulverization of the bone on the dorsal side 19 of the radius 11. The fracture 15 as shown may also result in loss of radial height, i.e., loss of height of the bone on the radial side 13 of the radius. In addition, the fracture 15 as shown may result in loss of volar tilt, i.e., loss of tilt of the bone towards the volar side 20 of the radius 11. Further, the fracture 15 as shown may result in radial shift, i.e., shift of the bone towards the radial side 13 of the radius 11. Moreover, the fracture 15 as shown may result in shortening of the radial column.

Conical Screw Embodiment

In one embodiment, the reduced bone fracture fixation device is configured as a conical screw. The body of the device can be generally in the shape of a cone, i.e., it can generally taper from a wider diameter at the proximal end of the device to a narrow diameter (e.g., a point) at the distal end of the device. In some instances, the conical screw does not have a “head”, that is, the widest portion of a standard screw that engages with a screwdriver (i.e., the conical screw is a headless screw). The body of the conical screw embodiment can be solid or it can be at least partially hollow. As such, at least a portion of the interior volume of the screw may be void space. For example, the device can have an internal passageway through at least a portion of the length of the device, e.g., the distal end of the device. In some instances in which the screw is hollow, the screw is cannulated, such that an internal passageway is defined inside of the screw from one end of the screw to the other, i.e., the screw is tubular. In a cannulated embodiment, the device can have an internal passageway extending the length of the device, e.g., from the proximal portion of the screw to the distal point of the screw.

In embodiments in which the conical screw has an internal passageway or opening, the opening forms a “wall”, i.e., the portion of the conical screw that surrounds the opening. In some embodiments the wall of the screw is continuous around the entire circumference of the conical screw. In some embodiments, one or more portions of the wall can have cut-outs such that the wall has “windows”, or openings, where there is communication between the internal passageway of device and the outside of the body of the device through the opening in the wall. The cut-outs can be any suitable shape (e.g., oval, ellipse, rectangle, etc.) or size, and can have any orientation with respect to the long axis of the device (e.g. longitudinal, diagonal, transverse, etc.)

In other embodiments, the wall of the conical screw is discontinuous at the proximal end of the device, e.g., the wall is formed of one or more segments. In some embodiments, therefore, the conical screw can have one or more extensions, or arm segments.

By “arm segment” is meant a proximal extension of a portion of the body of the device from the distal end of the device. In some embodiments, the subject device can have two or more arm segments, or three or more, or four of more, five or more, etc. The arm segments of a subject device can be separated by various distances. The arm segments can also be of various lengths, which can depend on the overall size and configuration of the device. In some embodiments, one or more individual arm segments can have cut-outs that provide “windows”, or openings formed within them, such that there is communication between the internal passageway of device and the outside of the body of the device through the opening in the arm segment.

For embodiments in which the reduced bone fracture fixation device includes one or more arm segments, the device may further include a first locking element, e.g., a hook, loop, or other feature, on a first arm segment at the proximal end of the device. The device may further include a second locking element, e.g., a hook or other feature, on a second arm segment at the proximal end. The first locking element and the second locking element may be configured to lock together, to constrain a maximum distance between the first arm segment and the second arm segment.

The reduced bone fracture fixation devices can also include one or more bone securing elements. By “bone securing element” is meant an element configured to secure the reduced bone fracture fixation device to bone at the surface of a reduced bone fracture or fracture void, such that the position of the reduced bone fracture device is maintained once it has been placed into the reduced fracture site (in other words, the device retains its position following placement). The bone securing element can be, for example, one or more threads on the outside surface of the device, such as threads on a screw; the bone securing element can also be barbs; hooks; loops; bumps; spurs; footholds; knuckles; coils, anchors; or other features etc.

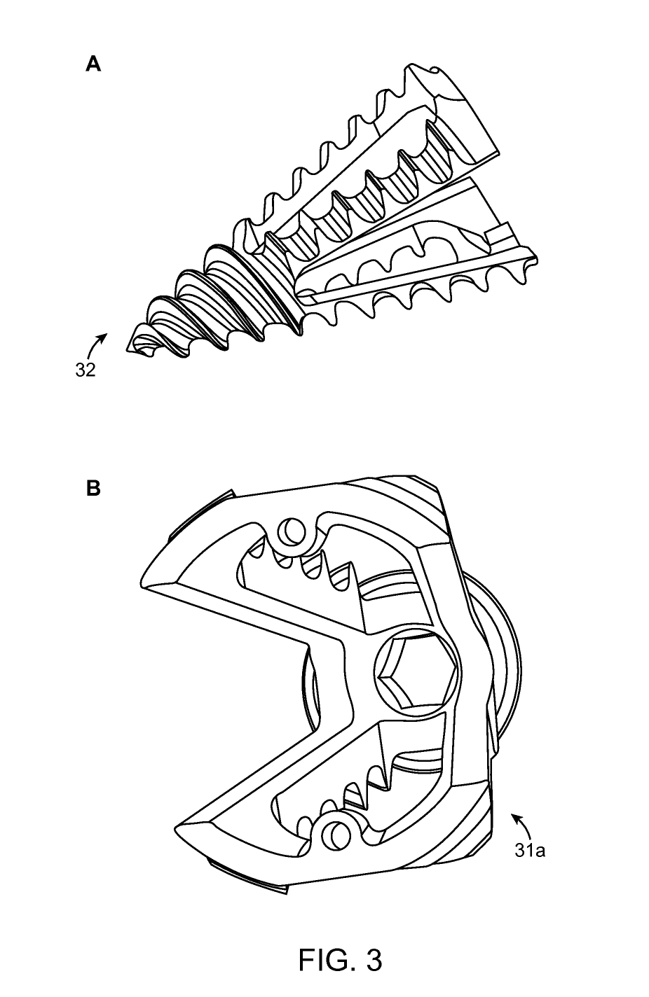

In one embodiment of the conical screw configuration, the bone securing element is a spiral thread or ridge that extends along at least a portion of the external surface of the conical screw device. In some instances, the pitch of the thread can be the same, and in some instances, the pitch of the thread may be variable. In these instances, the pitch of the thread (i.e., distance between the threads) may vary as desired. In some instances, the pitch ranges from 1 mm to 1 cm, such as 1.5 mm to 5 mm and including 2 mm to 3 mm. In some embodiments, the pitch may be variable along the length of the screw, e.g., the pitch may be smaller at the distal end, and widen at the proximal end. In some embodiments, the pitch can be single or double start thread. The thread or ridge can in some instances have a sharp peak, as shown in FIG. 3, and can also be flattened at the peak, or the outermost portion of the thread. The thread can be flattened over all or only a portion of the screw (e.g., the thread may be flattened at the proximal, or larger end of the screw). The flattening can result in an essentially planar configuration at the peak of the thread or ridge, or the flattening can result in a decrease in height of the thread, or both. The amount of flattening can be variable, e.g., flattening can involve 5% of the height of the thread, 10%, etc.

The subject device can also have various configurations between the threads, e.g., the ‘valley’ or grooves between the threads can be in a V-shape, or a U-shape, etc. Additionally, the depth of the “valley” or conversely, the height of the thread or ridge can also vary along the length of the screw embodiment. For example, the grooves can be shallower at the proximal end in order to provide more surface for contact with the surrounding bone.

The device can have any suitable diameter. In some instances, the diameter ranges from 3 mm to 3 cm, such as from 5 mm to 2.5 cm, or 5 mm to 2 cm. Additionally, the dimensions of the device can vary from one end of the device to the other, e.g., as for example with a conical screw embodiment. In some instances, the length of the device ranges from 3 mm to 6 cm, such as 4 mm to 5 cm, and including 8 mm to 4 cm.

The subject devices can in some embodiments be configured to be delivered to a reduced bone fracture site of interest using one or more tools, such as a guidewire, a grasping tool, a screwdriver, etc. For example, in a cannulated or hollow conical screw embodiment, the device can be deployed to a fracture site using a guidewire. In another example, the device can be positioned in a fracture site by using a tool such as a screwdriver adapted for use with the device (e.g. a hex head, cruciate, Phillips, Torx, or other three-dimensional locking method).

FIG. 3 illustrates an embodiment in which the reduced bone fracture fixation device is configured as a conical screw comprising a distal point 32, and two proximal arm segments 31a and 31b. Each arm segment further includes an opening, which allows communication between the internal portion of the device and the external portion of the device. In this embodiment, there is a bone securing element in the form of an external thread originating at the distal point 32, which continues on the outer surfaces of the two arm segments 31a and 31b. In this embodiment, the conical screw is hollow, with an opening at the distal point 32. In this embodiment, the conical screw can accept a hexagonal head or star screwdriver to allow rotational insertion. In this embodiment, the proximal end of the proximal arm segments contain small holes to accept a pincer compressing clamp to “close down” the proximal arms for easy removal of the device if required.

The angle of the conical screw, i.e., the angle formed between the distal point of the cone and the sides or walls of the cone can vary, e.g., in some embodiments the cone can be wider, or have a larger angle, and in other embodiments the cone will be narrower, or have a smaller angle. Accordingly, the volume of the internal open space of the conical screw embodiment will vary with changes in the above described angle. The volume of the internal open space will vary as well as with changes in length of the device, width of the internal passageway, etc.

Wire Embodiments

In another embodiment, the reduced bone fracture fixation device can have a configuration suitable for construction from wire. Wire embodiments of interest include, but are not limited to, those embodiments disclosed in PCT application Serial No. PCT/US10/24753, the disclosure of which is herein incorporated by reference. The wire can, in some embodiments, be formed into loops, coils, hinges, curves, pivots, or be curved or shaped to form a cage of various shapes, etc., or a combination thereof. The shape of the body of the device can therefore be any three-dimensional shape, such that the device can be successfully positioned in a reduced bone fracture. The shape of the subject devices can include, but is not limited to, a trapezoidal, pyramidal, cylindrical, conical, oblong, ovoid, cuboid, rectangular prism, triangular prism, or hexahedral shape, one or more “V” shapes, an “umbrella shape”, etc. The shapes may be symmetric or asymmetric, and in some embodiments a reduced bone fracture fixation device can include more than one shape or can vary in shape.

The cross-sectional configuration of the wire can be any suitable shape, such as round, oval, rectangular, square, etc. The wire can be of any diameter, which can range from 0.2 mm to 4 mm, such as from 0.3 mm to 3 mm, or 0.5 mm to 2.5 mm diameter. The wire may also vary in diameter along the length of the wire, such that the wire can have a smaller or larger diameter in some portions of the wire. For example, the wire may have a reduced or smaller diameter along one or more portions of an arm segment, etc. In some embodiments, the device may be formed of a continuous wire. In other embodiments, the device may be formed of more than wire. The overall dimensions for a reduced bone fracture fixation device for use in a reduced fracture site may have a length, or longest axis, which can range from 3 mm to 6 cm, such as 4 mm to 5 cm, and including 8 mm to 4 cm. Additionally, the dimensions of the device can vary from one end of the device to the other, e.g., as for example with a triangular prism embodiment. As discussed above, the actual dimensions of the reduced fracture fixation device will vary according to the size of the fracture, the size and extent of the fracture site, etc.

In some embodiments, the subject devices constructed from wire can have one or more extensions, or arm segments, i.e., a portion of the body of the device that extends proximally from the distal end of the device. As discussed above, the subject device can have two or more arm segments, or three or more, or four or more, etc. The arm segments of a subject device can be separated by various distances. The arm segments can also be of various lengths, which can depend on the overall size and configuration of the device.

For embodiments in which the reduced bone fracture fixation device includes one or more arm segments, the device may further include a first locking element, e.g., a hook, loop, anchor, or other feature, on a first arm segment at the proximal end of the device. The device may further include a second locking element, e.g., a hook, loop, anchor, or other feature, on a second arm segment at the proximal end. The first locking element and the second locking element may be configured to lock together to constrain a maximum distance between the first arm segment and the second arm segment. The locking elements can in some instances be joined together by an additional process, such as welding, or laser welding. The locking elements can be integral to the arm segments, or they can be separate elements.

The reduced bone fracture fixation devices configured from wire can also include one or more bone securing elements, i.e., an element configured to secure the fixation device to bone at the surface of a reduced bone fracture. The bone securing element can be, for example, barbs; hooks; loops; bumps; spurs; footholds; knuckles; coils, anchors; or other features etc. The bone securing elements can be located in any suitable location on the body of the device. In one embodiment, the bone securing element can located at the proximal end of one or more arm segments.

The reduced bone fracture fixation devices can also include one or more fastening elements. By “fastening element” is meant an element configured to maintain the reduced bone fracture fixation device in an un-deployed configuration prior to positioning the device in a reduced fracture site. The fastening element can also, in some instances, function to maintain the device in a deployed configuration (e.g., by locking an expanded coil in the expanded configuration). In some embodiments, the device can include a release mechanism. By “release mechanism” is meant an element that releases the fastening element to allow the device to deploy (e.g., expand). The release mechanism can be, for example, a button, a switch, etc. A fastening element can be secured to the reduced fracture fixation device, such that the fastening element can be released to allow the reduced fracture fixation device to expand once it has been placed into the reduced fracture site. In some instances, the fastening element can then lock the expanded device in place. The fastening element may be formed integrally with the device, or the fastening element may be a separate element. The fastening element can be any suitable shape, such as a rectangular prism, a clip, a hook, a clasp, a band, etc.

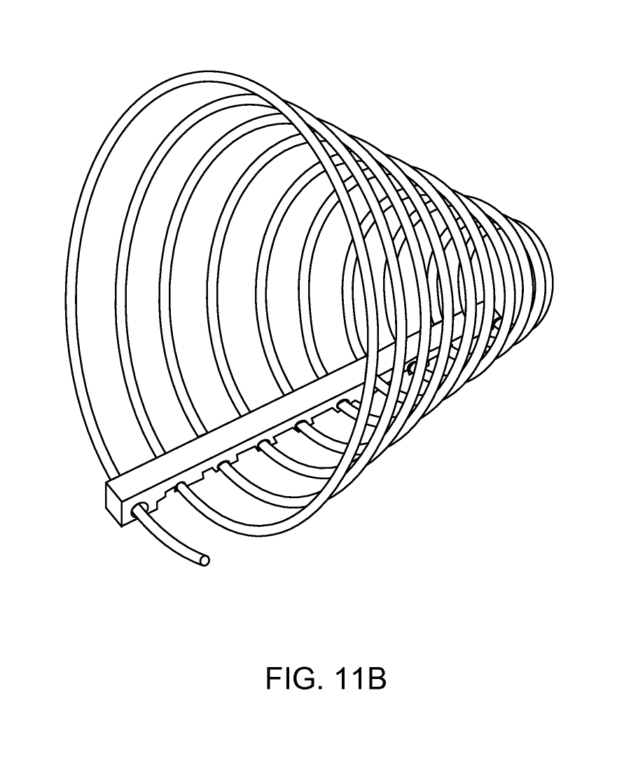

As an example, the reduced fracture fixation device may be in the shape of a small-diameter cylindrical coil, and when a fastening mechanism is released, the fixation device may expand into a larger diameter conical shape (see, e.g., FIGS. 11A-B). In another example, the reduced fracture fixation device may be in a linear configuration, and when a fastening mechanism is released, the fixation device may expand into an “umbrella” configuration. In some embodiments, the reduced fracture fixation devices may include more than one fastening element.

The subject devices can be configured to be delivered to a reduced bone fracture site of interest using one or more tools, such as a grasping tool, or a forceps, a guidewire, etc.

According to one embodiment of the present invention, a reduced fracture fixation device may include a resilient hinge, a first arm extending from one end of the hinge, and a second arm extending from another end of the hinge, in which each of the hinge, the first arm, and the second arm includes at least one loop formed from wire. The hinge may be configured to bias the first arm and the second arm away from each other, such that the two arm elements are configured to apply opposing forces on bone of the reduced bone fracture. The hinge and the first and second arm segments can be formed from a single wire, or from multiple wires joined together. A plane defined by the at least one loop of the first arm segment may be substantially parallel to a plane defined by the at least one loop of the second arm segment. A plane defined by the at least one loop of the hinge may be substantially perpendicular to each of a plane defined by the at least one loop of the first arm segment and a plane defined by the at least one loop of the second arm segment. The hinge, the first arm, and the second arm together may substantially form one of a trapezoidal, pyramidal, triangular, conical, oblong, and ovoid shape. In some embodiments, the subject device is not the device disclosed in PCT application Serial No. PCT/US10/24753.

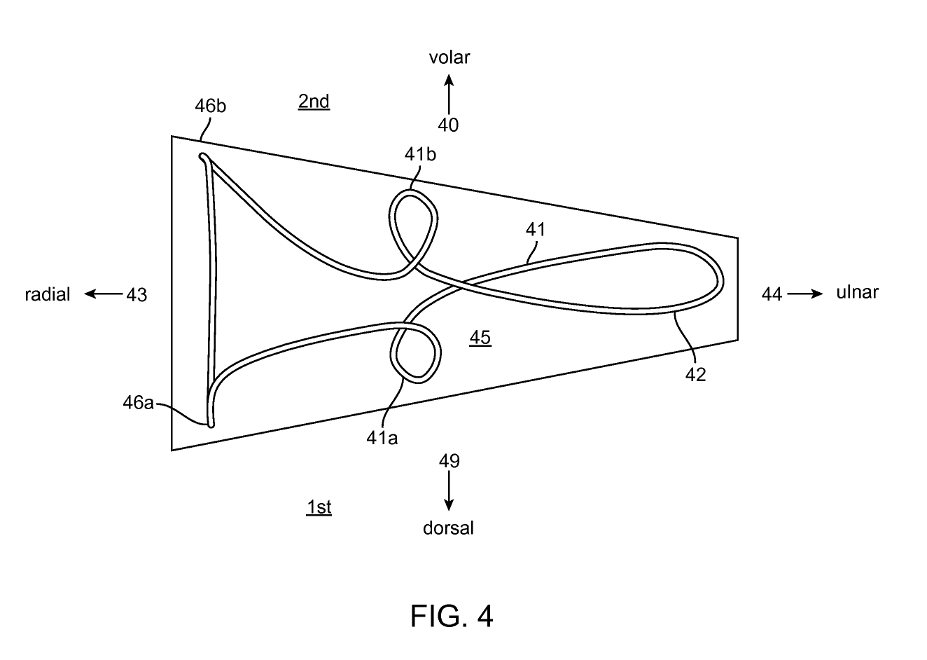

FIG. 4 illustrates a schematic view of one embodiment of a reduced bone fracture fixation device 41 located within the metaphyseal void created by bone fracture 45. The reduced bone fracture fixation device 41 includes a resilient hinge 42 including at least one loop, a first arm segment 41a including at least one loop and attached to one end of the hinge 42, and a second arm segment 41b including at least one loop and attached to the other end of the hinge 42. The hinge 42 at the distal end of the device is positioned on the ulnar side 44 of the fracture 45. The first arm segment 41a is positioned at the proximal edge of the fracture 45. The second arm segment 49 is positioned at the distal edge of the fracture 45.

The subject device as shown in FIG. 4 may also include one or more bone securing elements 46 at the proximal end of the device, i.e., at the end of first arm segment 41a and second arm segment 41b opposite distally located hinge 42. Bone securing elements 46 can attach to opposite sides of the fracture 45, e.g., a distal side of the fracture, and a proximal side of the fracture. In the example of the distal radial fracture of a wrist, distal side refers to the side nearer the hand, whereas the proximal side refers to the side nearer the elbow. In other embodiments, for example, one bone securing element 46a may attach on the dorsal side 49 of the fracture 45, and the other bone securing element 46b may attach on the volar side 40 of the fracture 45.

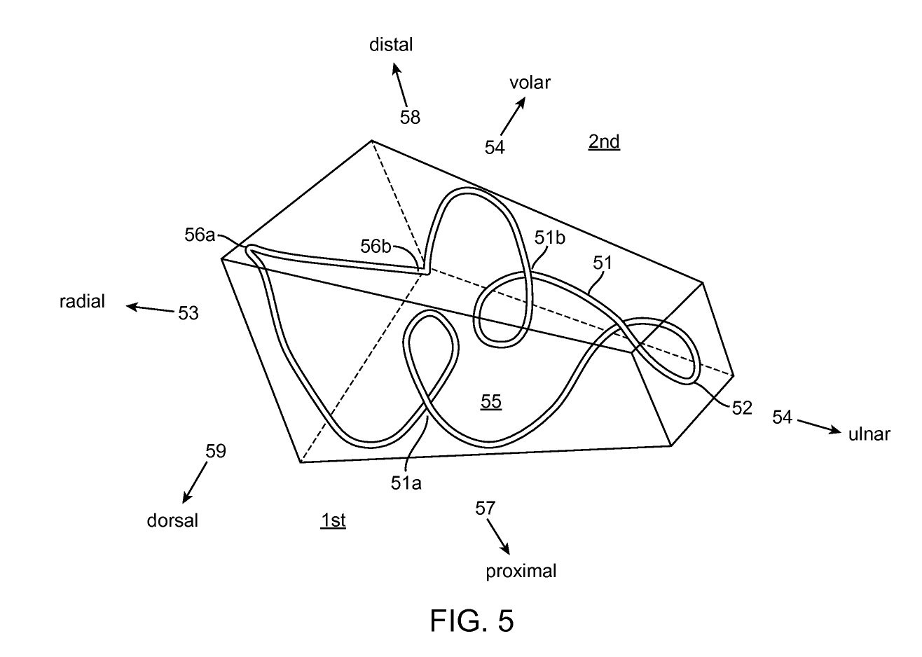

FIG. 5 illustrates a schematic perspective view of an embodiment of a reduced bone fracture fixation device 51 situated within the metaphyseal void created by bone fracture 55. The subject device 51 includes a resilient hinge 52 including at least one loop, a first arm segment 51a including at least one loop, and a second arm segment 51b including at least one loop. The hinge 52 is situated on the ulnar side 55 of the fracture 55. The first arm segment 51a is situated on the dorsal side 59 of the fracture 55. The second arm segment 51b is situated on the volar side 50 of the fracture 55.

The bone fracture fixation device 51 as shown in FIG. 5 may also include a first bone securing element 56a at a proximal end of the first arm segment 51a opposite hinge 52. The device 51 may also include a second bone securing element 56b at a proximal end of the second arm segment 51b opposite hinge 52. The bone securing elements 56a and 56b can attach to bone on opposite sides of the fracture 55, e.g., one bone securing element 56a can attach on the dorsal surface of the fracture, and the other bone securing element 56b can attach on the volar surface 50. One bone securing element 56a can also attach on the distal surface 58 of the fracture, and the other bone securing element 56b can attach on the proximal surface 57, or vice versa.

As shown in FIGS. 4 and 5, the reduced bone fracture fixation devices may form a substantially trapezoidal shape, or a substantially pyramidal shape. As discussed above, the device 41 or 51 may form other shapes, e.g., triangular, conical, oblong, ovoid, or others. Further, although the embodiments of FIGS. 4 and 5 include only a single loop for each of the hinges 42 or 52, the first arm segment 41a or 51a, and the second first arm segment 41b or 51b, there can be more than one loop in one or more of these elements. For example, the hinge can include a single loop, and the first and second arm segments can each include a double loop, etc.

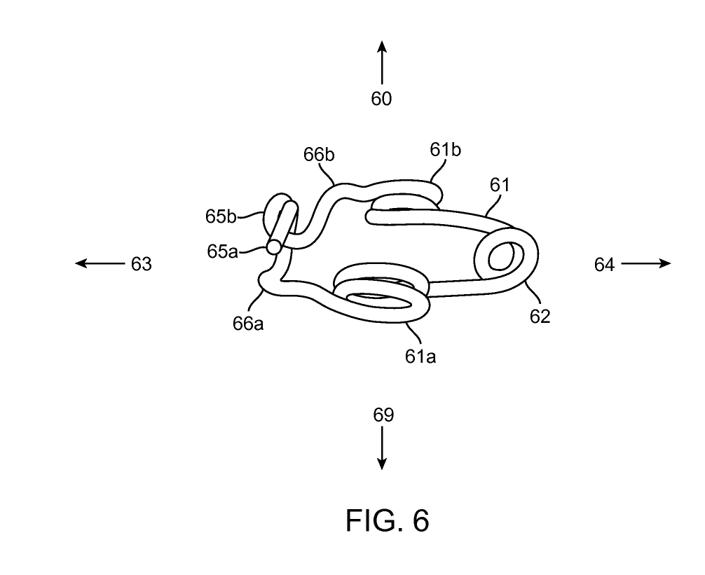

FIG. 6 illustrates a view of a reduced bone fracture fixation device 61. Similar to the example embodiment shown in FIGS. 4 and 5, the embodiment of FIG. 6 includes a hinge 62, a first arm segment 61a, a second arm segment 61b, a first bone securing element 66a, and a second bone securing element 66b. The hinge 62 is situated on the ulnar side 64 of the fracture, the first arm segment 61a is situated on the dorsal side 69 of the fracture, and the second arm segment 61b is situated on the volar side 60 of the fracture.

In the embodiment shown in FIG. 6, the hinge 62 includes a single small loop, the first arm segment 61a includes a double large loop, and the second arm segment 61b includes a double medium loop. The single small loop of the hinge 62 may be sized to fit within the ulnar side 64 of the fracture. Based on the exemplary distal radial fracture illustrated in FIGS. 1 and 2, the fracture on the dorsal side 69 is larger relative to the fracture on the volar side 60. Thus, the double medium loop of the second arm segment 61b may be sized to fill the volar side 60 of the fracture, and the double large loop of the first arm segment 61a may be sized to fill the relatively larger dorsal side 69 of the fracture.

Although the embodiment illustrated in FIG. 6 includes either a single loop or double loops, it is understood that a variable number of loops may be utilized. The number of loops may be chosen to provide the desired spring effect and/or potentially better interference fit within the fracture. For example, an increase in the number of loops may increase the spring effect and/or improve the interference fit of the device. Moreover, the material of the device and the thickness or gauge of the wire may be varied to achieve the desired spring effect and/or interference fit as well.

The hinge 62 may be configured such that the first arm segment 61a and the second arm segment 61b are biased away from each other. By this mechanism, the device 61 may provide additional tension to the fracture and sufficiently fill the void, thereby acting as a three-dimensional reduction device and providing load-sharing. Because the reduced bone fracture fixation device 61 itself acts as the reduction device, additional bone grafts or filler substitutes to fill the metaphyseal void may be unnecessary.

Further, the embodiment shown in FIG. 6 includes a first locking element 65a and a second locking element 65b. The first locking element 65a is situated on the first arm segment 61a at an end opposite the hinge 62, and the second locking element 65b is situated on the second arm segment 61b at an end opposite the hinge 62. The two locking elements 65a and 65b may be configured to lock together to constrain a maximum distance between the first arm segment 61a and the second arm segment 61b. For example, the locking elements 65a and 65b may function similarly to a safety pin (i.e., locking the arm segments that are biased away from each other by the resilient hinge creates potential energy in the two arm segments). In addition, the locking elements 65a and 65b may be formed as hooks or any other shapes that may interlock or constrain relative movement.

When locked together, the two locking elements 65a and 65b form an oblique radial strut between the bone securing elements 66a and 66b. That is, the bone securing elements 66a and 66b and the locking elements 65a and 65b may form a strut that traverses the radial side 63 of the fracture, e.g., from a dorsal, distal side of the fracture (e.g., at location 56a in FIG. 5) to a volar, proximal side of the fracture (e.g., at location 56b in FIG. 5). Alternatively, the strut may traverse the radial side 63 of the fracture, e.g., from a dorsal, proximal side of the fracture to a volar, distal side of the fracture.

As shown in the exemplary embodiment of the reduced bone fracture fixation device 61 of FIG. 6, a plane formed by the at least one loop of the first arm segment 61a may be substantially parallel to a plane formed by the at least one loop of the second arm segment 61b. In addition, a plane defined by the at least one loop of the hinge 62 may be substantially perpendicular to each of the plane defined by the at least one loop of the first arm segment 61a and the plane defined by the at least one loop of the second arm segment 61b. This configuration can provide sufficient tension to fill the fracture void and maintain sufficient reduction of the fracture.

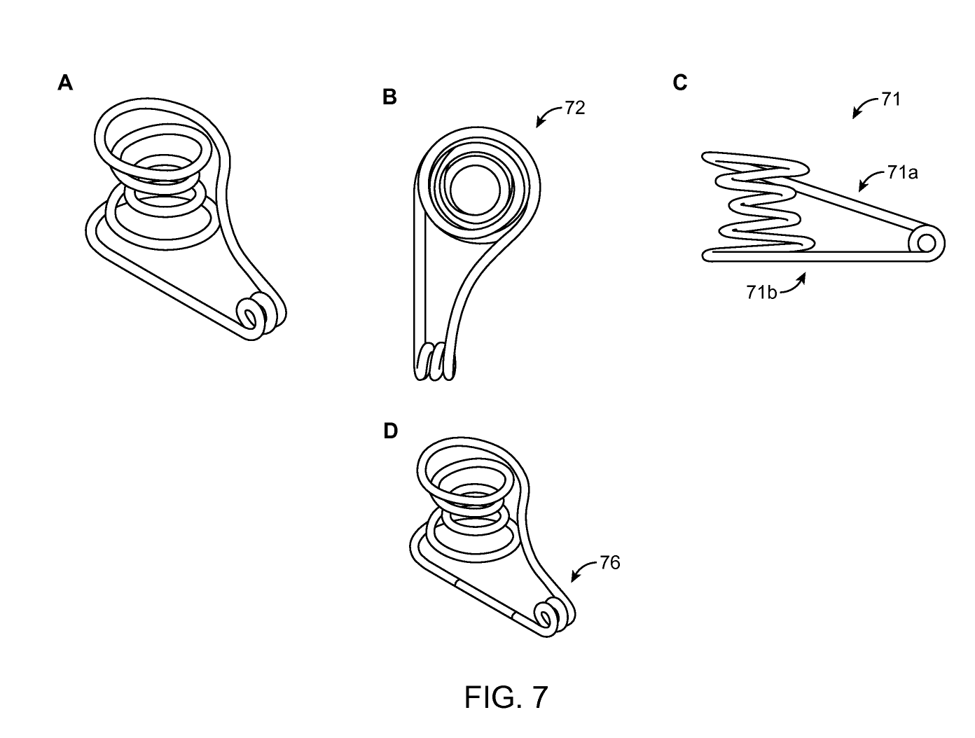

FIGS. 7A-D illustrate four perspective views of a continuous wire embodiment of a reduced bone fracture fixation device. In this embodiment, first arm segment 71a is continuous with a coil 76 on one side, and hinge 72 and the proximal end of second arm segment 71b on the other side. Hinge 72 in this embodiment is formed of multiple loops of a varying diameter coil at the proximal end of the subject device 71. In this embodiment, a hollow locking tube, or sleeve, is integrated to cover the two ends of the spring wire (linking the two arm segments) to allow some stress motion yet maintain stability of the device long term (light area in FIG. 7D).

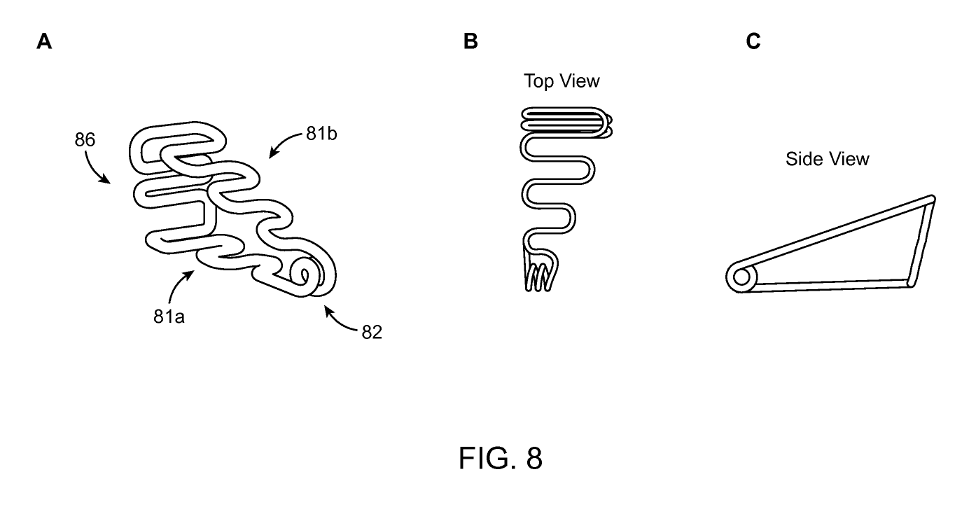

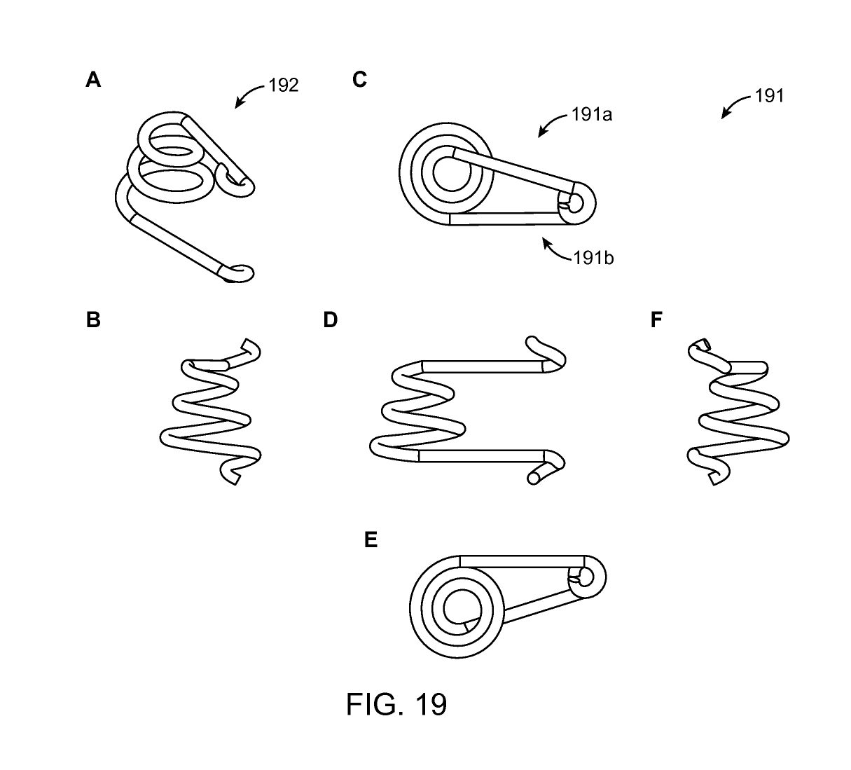

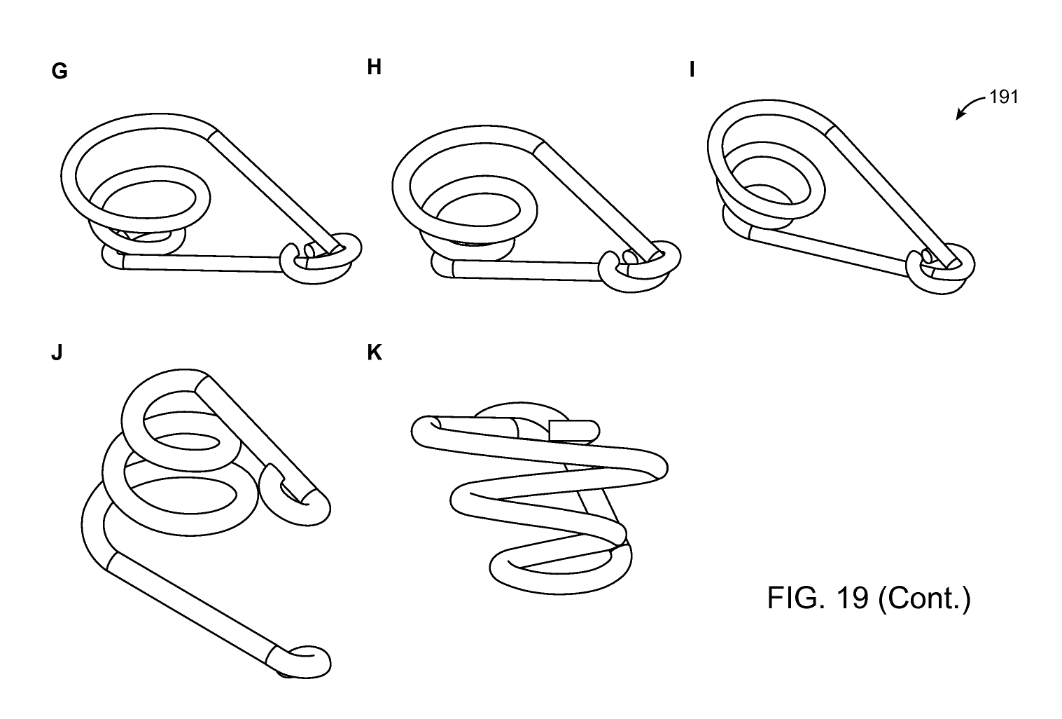

FIGS. 19A-K illustrate multiple perspective views of another continuous wire embodiment of a reduced bone fracture fixation device. In this embodiment, the hinge 192, first arm segment 191a and a second arm segment 191b are continuous. Hinge 192 in this embodiment is formed of multiple loops of a varying diameter coil at the proximal end of the subject device 191, with a major diameter on one side, and a minor diameter on the other side of the hinge. In this embodiment, the distal portions of the two arm segments are formed with integral partial loops or hooks (i.e., locking elements) that are configured to lock together (FIGS. 19G-I). The loops can in some instances be laser welded together in a side-to-side manner, such as at the area of the device labeled “engagement”, as shown in FIG. 19 G. FIGS. 8A-C show top, side, and angled perspective views of an embodiment of the subject device with a pivot coil configuration. In this embodiment, first and second arm segments 81a and 81b are continuous with a segment 86 linking the proximal end of first arm segment 81a and the proximal end of second arm segment 81b. Segment 86 in this embodiment is formed of a wire with multiple pivots of varying width, resulting in parallel segments of wire separated by various distances. First arm segment 81a and second arm segment 81b are also formed of a wire with multiple pivots.

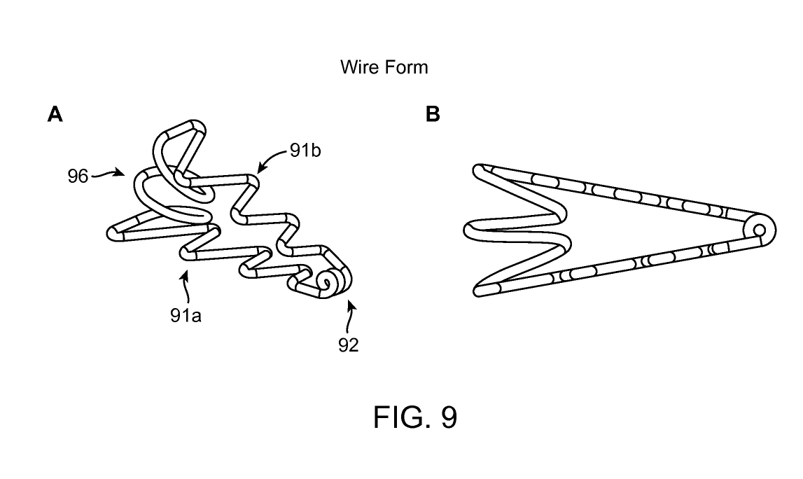

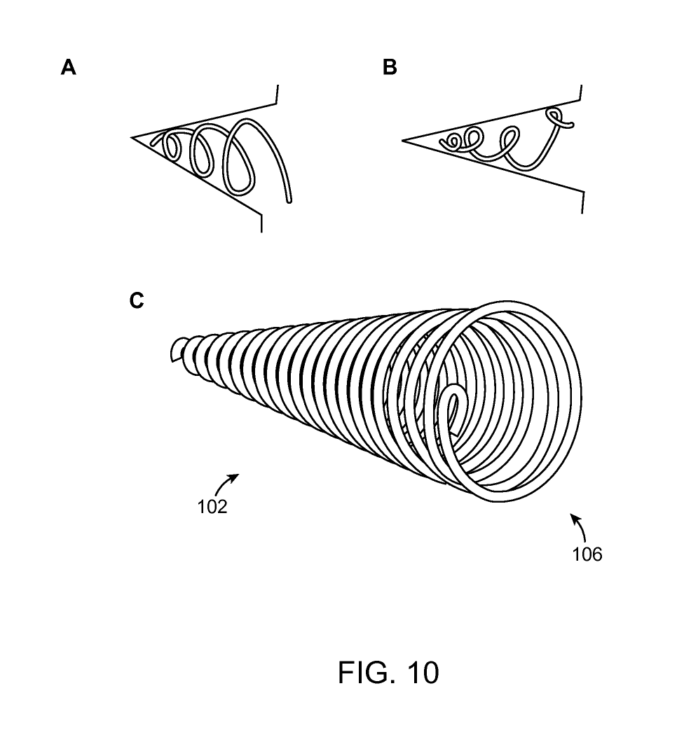

In another embodiment, FIGS. 9A-B show two perspective views of another embodiment of a reduced bone fracture fixation device, in which the proximal ends of arm segments 91a and 91b are linked by a wire segment 96 formed of several loops at the proximal end of the subject device. First arm segment 91a and second arm segment 91b are also formed of a wire with multiple pivots.

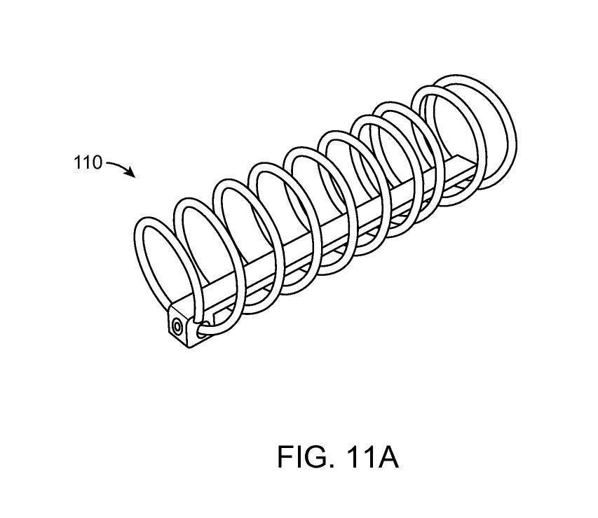

An embodiment in which the reduced bone fracture fixation device is configured as a conical cage formed of wire is shown in FIG. 10. In this embodiment, the subject device is configured as a spring coil, as shown within a schematic fracture void in FIG. 10A. In some instances, the device can be configured as a double coil, as shown in FIG. 10B. The subject device shown in FIG. 10C comprises a conical coil with a distal end 102 which expands to a larger diameter proximal end 106 of the device. In this embodiment, the entire length of the device can be configured to apply tension on bone of the reduced bone fracture.

As discussed above, the reduced bone fracture fixation device can also be configured to have both an un-deployed configuration, prior to being placed in the target fracture site, and a deployed configuration once the device is positioned in the fracture site. For example, FIG. 11A illustrates an un-deployed reduced bone fracture fixation device configured as a cylindrical cage formed of wire. In this embodiment, the subject device also includes a fastening element 110. The fastening element in this instance is configured as a longitudinal deployment guide mechanism which maintains the subject device in an un-deployed configuration, e.g., a cylindrical configuration. The fastening element can include a release mechanism, such that the fastening element can be released to allow subsequent predetermined deployment of the device to expand to a deployed configuration, e.g., a conical configuration, once it has been placed into the reduced fracture site. In some instances, the fastening element can be used to lock the deployed device in place with e.g., a set screw. This embodiment is shown in FIG. 11B. In this embodiment, the entire length of the device can be configured to apply tension on bone of the reduced bone fracture.

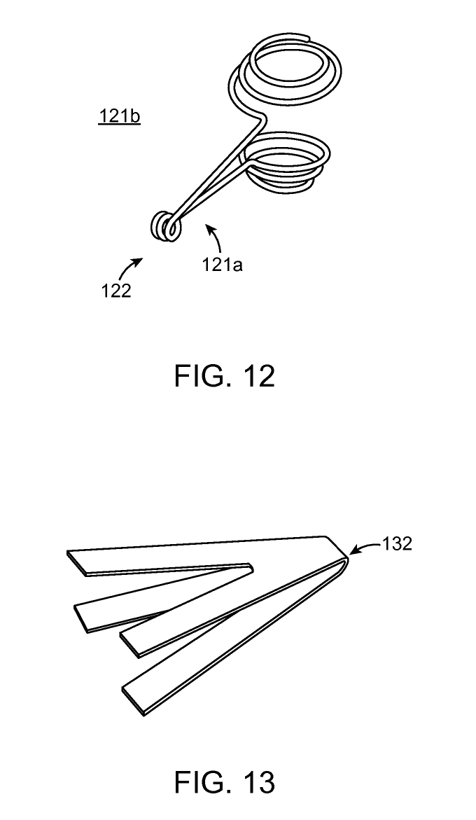

The embodiment shown in FIG. 12 shows a device formed of wire which includes a distal coiled hinge 122, with a first arm segment 121a and a second arm segment 121b. In this embodiment, both the first and second arm segments include a coiled proximal end, configured to exert opposing forces on bone surfaces of the fracture site sufficient to maintain fracture reduction.

Planar Element Embodiments

In another embodiment, the reduced bone fracture fixation device can have a configuration suitable for construction from a planar element. By “planar element” is meant a flat, essentially two-dimensional element, such as a ribbon or sheet. The shape of the subject devices can include, but is not limited to, one or more “V” shapes, a trapezoidal, pyramidal, cylindrical, conical, oblong, ovoid, cuboid, rectangular prism, triangular prism, or hexahedral shape, an “umbrella shape”, etc. The shapes may be symmetric or asymmetric, and in some embodiments a reduced bone fracture fixation device can include more than one shape or can vary in shape. The planar element can also be curved, e.g., in a concave or convex direction, or can be curved or shaped to form a cage of various shapes as disclosed above. In some embodiments, the device may be formed of one or more planar elements, or a combination of one or more planar elements and one or more wire elements.

The shape of the body of the device can therefore be any suitable three-dimensional shape, such that the device can be successfully positioned in a reduced bone fracture. The planar element can be any width, which can range from 3 mm to 6 cm, such as from 4 mm to 4 cm, or 5 mm to 3 cm. In some embodiments, the device may be formed of a continuous planar element. In other embodiments, the device may be formed of more than planar element joined together. The overall dimensions of the device can have a length, or longest axis, which can range from 3 mm to 6 cm, such as 4 mm to 5 cm, and including 8 mm to 4 cm. Additionally, the dimensions of the device can vary from one end of the device to the other, e.g., as for example with a “V”-shaped embodiment. As discussed above, the actual dimensions of the reduced fracture fixation device will vary according to the size of the fracture, the size and extent of the fracture site, etc.

In some embodiments, the subject devices constructed from a planar element can have one or more extensions, or arm segments, i.e., a portion of the body of the device that extends proximally from the distal end of the device. As discussed above, the subject device can have two or more arm segments, or three or more, or four or more, etc. The arm segments of a subject device can be separated by various distances. The arm segments can also be of various lengths, which can depend on the overall size and configuration of the device.

For embodiments in which the reduced bone fracture fixation device includes one or more arm segments, the device may further include a first locking element, e.g., a hook or other feature, on a first arm segment at the proximal end of the device. The device may further include a second locking element, e.g., a hook or other feature, on a second arm segment at the proximal end. The first locking element and the second locking element may be configured to lock together to constrain a maximum distance between the first arm segment and the second arm segment.

The reduced bone fracture fixation devices configured from wire can also include one or more bone securing elements, i.e., an element configured to secure the fixation device to bone at the surface of a reduced bone fracture. The bone securing element can be, for example, barbs; hooks; loops; bumps; spurs; footholds; knuckles; coils, anchors; or other features, etc. The bone securing elements can be located in any suitable location on the body of the device. In one embodiment, the bone securing element can located at the proximal end of one or more arm segments.

The reduced bone fracture fixation devices can also include one or more fastening elements, i.e., an element configured to maintain the reduced bone fracture fixation device in an un-deployed configuration prior to positioning the device in the reduced fracture site. The fastening element can also, in some instances, function to maintain the device in a deployed configuration (e.g., by locking an expanded device in the expanded configuration). In some embodiments, the device can include a release mechanism, e.g., a button, a switch, etc. A fastening element can be secured to the reduced fracture fixation device, such that the fastening element can be released to allow the reduced fracture fixation device to expand once it has been placed into the reduced fracture site. The fastening element may be formed integrally with the device, or the fastening element may be separate. The fastening element can be any suitable shape, such as a rectangular prism, a clip, a hook, a clasp, a band, etc.

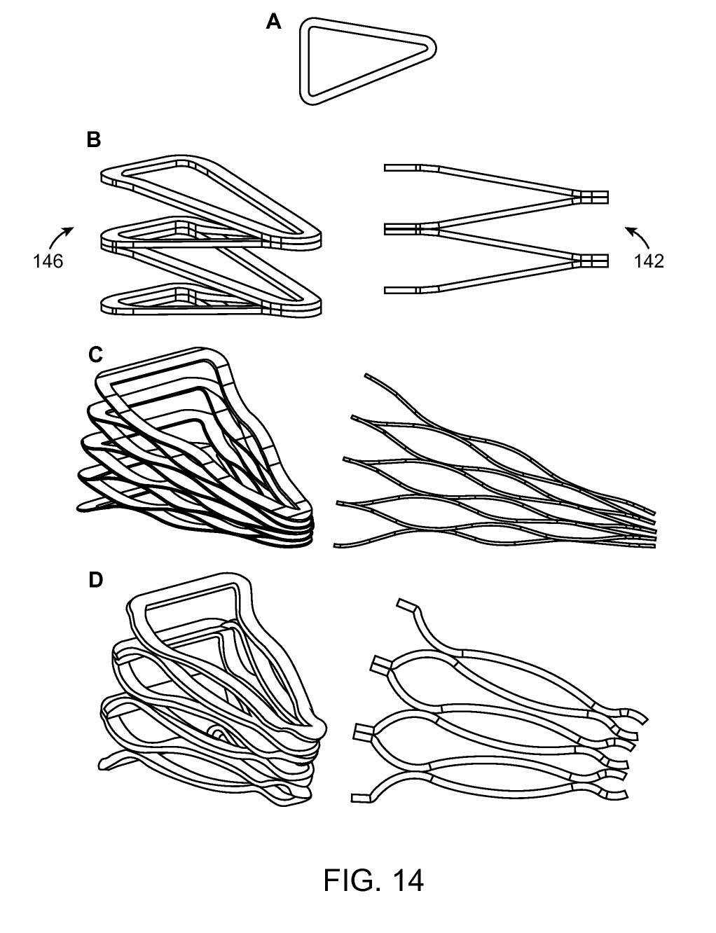

As an example, the reduced fracture fixation device may be in the shape of a flat triangle formed by one or more planar elements, such that the triangle has a central opening (see, e.g., FIG. 14A). The device may be formed of multiple layers of the triangular planar elements. The fixation device may expand into a shape similar to a quadrilateral pyramid (see, e.g., FIGS. 14B-D), wherein the proximal end of the device expands outward from the distal end of the device. In some embodiments, the reduced fracture fixation devices may include more than one fastening element.

The subject devices can be configured to be delivered to a reduced bone fracture site of interest using one or more tools, such as a grasping tool, plier, clip, forceps, hollow tube cannula, introducer, plunger, sled, and other assistive devices that may deliver, deploy, and/or protect soft tissues in the delivery of the device.

FIG. 13 illustrates an embodiment of a reduced bone fracture fixation device formed of a planar element, e.g. a flat ribbon or sheet. The body of the device shown in FIG. 13 includes four arm segments, similar to a four-legged tripod, or two “V” shapes connected by a fold at distal end 132.

Additional embodiments of reduced bone fracture fixation devices formed from essentially planar elements are shown in FIGS. 14A-D. The subject devices shown in FIGS. 14B-D are formed from one or more triangular planar elements, e.g. a flat ribbon in the shape of a triangle, as shown in FIG. 14A. The embodiment depicted in FIG. 14B includes four triangular planar elements, which may be integral or formed from one or more separate elements. The triangular planar elements in FIG. 14B have one or two points of contact between each triangular planar element and the adjacent planar elements, at the proximal 146 and/or distal end 142 of the device (e.g., similar to a wave spring). The planar elements can be joined by any suitable manner, e.g., laser welded. FIG. 14C shows triangular planar elements with more than two points of contact between adjacent planar elements along the length of the device. In FIGS. 14C and 14D the embodiments shown are formed of multiple triangular elements in which the triangular planar elements are curved.

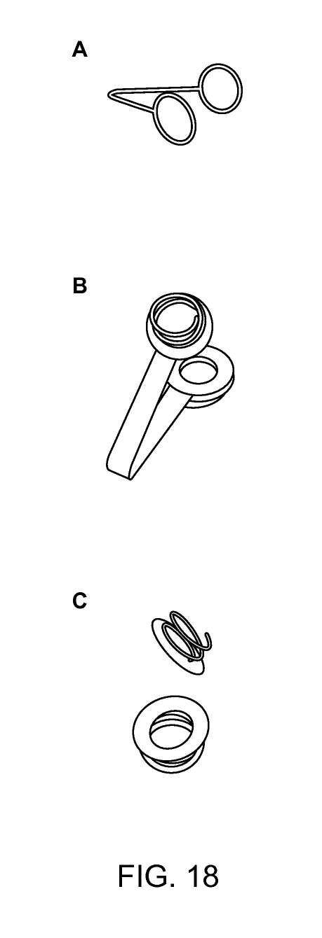

As discussed above, in some embodiments, the device may be formed of a combination of one or more planar elements and one or more wire elements. For example, FIG. 18 illustrates an embodiment in which the device has a “V” shape with two arm segments. In FIG. 18A, the device is formed of a wire, and the proximal portion of the arm segments is formed into a ring configuration. In FIG. 18B, the device is formed of a planar element, and the proximal portion of the arm segments can be in a linear configuration, or can be formed into a ring configuration. In both embodiments, any suitable configuration is possible (e.g., triangular, square, oval, etc). In this embodiment, the tension element formed by the two arm segments can provide a fixed amount of force exerted on bone of a reduced fracture. The amount of force exerted by this tension element can vary depending on the size and shape of the tension element, as well as the materials used, the thickness of the device (e.g., the thickness of the arm segments), etc.

In addition, the arm segments of the embodiments as shown in FIGS. 18A and B can be configured to mate with one or more interchangeable elements. By “interchangeable element” is meant an element that can mate with a portion of the body of a device of the subject invention, e.g., the proximal portion of an arm segment. One or more interchangeable elements can provide additional force on bone of a reduced bone fracture, e.g., can provide an additional tension element to the device. For example, a subject device can be configured as a wire embodiment with two arm segments, as in FIG. 18A, and an interchangeable element can be mated with the proximal portion of each of the two arm segments. In this instance, the wire embodiment with two arm segments can exert a fixed amount of force on bone of a reduced fracture, and the addition of two interchangeable elements can provide additional force, such that the combined force of the tension element formed by the two arm segments and the tension element provided by the two interchangeable elements on the bone of the reduced fracture is sufficient to maintain reduction of the reduced fracture.

In some instances, an interchangeable element can provide force on bone of a reduced bone fracture at an additional location on the bone. For example, a subject device can be configured as a planar embodiment with two arm segments, as in FIG. 18B, and an interchangeable element can be mated with a portion of each of the two arm segments (e.g., the proximal portion or a middle portion). In this instance, the planar embodiment with two arm segments can exert a fixed amount of force on one location of bone of a reduced fracture, and the addition of two interchangeable elements can provide additional force at an additional location on the bone (e.g., a location distal to the proximal portion of the arm segments), such that the combined force of the tension element formed by the two arm segments and the tension element provided by the two interchangeable elements on the bone of the reduced fracture is sufficient to maintain reduction of the reduced fracture.

An interchangeable element can be configured to mate with the body of a subject device by using a feature in the body of the device (e.g., the body of the device is configured with one or more slots, notches, grooves, etc.). In some instances, an interchangeable element can be configured to mate with the body of a device by a feature in the interchangeable element (e.g., the interchangeable element can have one or more clips, clasps, hooks, etc.). In some instances both the body of the device and the interchangeable element can be configured to mate by complementary features (e.g, a notch that fits into a slot, or protrusions that interdigitate, etc). In some embodiments, a separate attaching element can be used to mate one or more interchangeable elements to the body of the device (not shown). By “attaching element” is meant an element that securely mates one or more interchangeable elements with a body of the device (e.g., a clip, clasp, band, etc). In some instances, an interchangeable element can be configured to mate with the body of the device such that it overlaps with a portion of the body, e.g., the interchangeable element overlaps the proximal portion of an arm segment, such that the arm segment may remain essentially the same length. In other instances, an interchangeable element can be configured to mate with the body of the device such that it does not overlap with a portion of the body, e.g., the interchangeable element mates with the proximal end of an arm segment such that it extends the length of an arm segment. In some instances, an interchangeable element can be formed of more than one part, e.g., a planar ring and a coiled spring, as shown in FIG. 18C.

The interchangeable element can have any suitable configuration such that the interchangeable element can provide force on bone of a reduced bone fracture. The interchangeable element can therefore vary in shape, tension, or dimension. For example, the interchangeable element can be in the shape of a spring mounted on a flat washer, as shown in FIG. 18C. Other possible shapes include cylindrical or tapered coils, hinges, loops, etc. The interchangeable elements can vary in dimension (e.g., depending on the size of the device, the size of the fracture and/or the fracture location). The interchangeable elements can also vary in the amount of force exerted on bone of a reduced fracture by variations in the size and shape of the interchangeable element, as well as the materials used, the thickness of the element, etc.

The reduced bone fracture fixation devices described above can be made of a variety of biocompatible materials or metallic materials that combine strength, flexibility, and fatigue resistance. For example, the fixation device can be formed using at least one of autograft or allograft bone, stainless steel, titanium, a nickel-titanium alloy such as nitinol, a nickel-cobalt alloy, another cobalt alloy, a vanadium alloy, tantalum, chromoly steel or CRMO, PEEK 15 (polyaryletheretherketone), other biocompatible polymers and plastics, and combinations or mixtures thereof. In some embodiments, the reduced bone fracture fixation devices or any portion thereof can include shape memory materials, which are materials that have a temperature induced phase change, e.g., a material that if deformed when cool, returns to its “undeformed”, or original, shape when warmed.

In some embodiments, the fixation device may be coated with a substance, over the entire device or a portion of the surface of the device. In some embodiments, the coating can include a therapeutic agent (e.g., an antibiotic, an anti-inflammatory agent), an agent to promote osteo-integration (e.g., hydroxyapatite), a hardening agent (e.g., titanium nitride), an anodizing treatment, etc. In some embodiments, a fixation device may be coated with a combination of agents, e.g., antibiotics and anti-inflammatory agents; agents or features to promote osteo-integration, etc. In other embodiments, a fixation device may have more than one coating. For example, a reduced bone fracture fixation device can be coated with an antibiotic agent and also have an anodizing treatment applied to the device.

The subject devices can be used in conjunction with various fixation devices as known in the art, such as a K-wire, nails, screws, rods, staples, etc. The reduced bone fracture fixation devices can also be configured to be used with bone graft including autograft or allograft, osteoconductive or osteoinductive synthetic graft, Bone Morphogenetic Proteins (BMPs), or bone cements (e.g., calcium phosphate, calcium sulfate, etc.).

The subject devices and methods can be used in open surgical methods, such that the device may be configured to be implanted with minimal soft tissue invasion and bony disruption. The subject devices and methods can be also be used in minimally invasive surgical, or interventional procedures.

After implantation, the subject devices can maintain reduction of a fracture by acting as a cortical strut and a three-dimensional reduction device filling the metaphyseal void. The reduced bone fracture fixation device provides sufficient resistance to forces to maintain reduction of a fracture (e.g., compressive and torsional forces, which can pull the bone out of alignment during the fracture healing phase). In the case of a distal radius fracture, the healing process typically lasts from 4 to 8 weeks.

The reduced bone fracture fixation device can also provide load-sharing healing of the fracture site. Reduced and maintained fractures will heal even if some defect cavitation has occurred from the fracture itself, since the fracture has direct exposure to bone marrow elements with active bone cells (osteoblasts, osteoclasts, osteocytes, and other blood elements known to influence bone healing and remodeling) provided the fracture remains reasonably stable. Additionally, the subject devices do not require a second procedure for removal of the device, e.g., as required with conventional plate and screw fixation. However, these devices can be removed either in the acute setting due to unforeseen factors such as infection by direct manipulation and extraction. The devices can also be removed even in the setting of a healed fracture by either compression and unscrewing in the conical screw design, or controlled corticotomy with wireform compression, cutting, and removal. In the latter cases, the overall healed fracture integrity is maintain in a three-dimensional phase.

Further, the subject devices may include slight over-distraction of the arm segments such that slight compression by the surrounding soft tissues results when the device is implanted into a bone or fracture void, providing further resistance to motion.

### Methods

The subject devices find use in methods for repair of a bone fracture. The subject devices can be used in an open surgical procedure, a minimally invasive surgical procedure, or in some embodiments, an interventional procedure.

Methods of using a reduced bone fracture fixation device can include identifying a subject with a bone fracture, reducing the bone fracture, and then introducing into the reduced bone fracture a device comprising a body dimensioned to be positioned in the reduced bone fracture. The subject devices can be inserted into a bone or fracture void, such that body of the device cooperates to tension the bone or fracture void. For example, the reduced bone fracture fixation device may be configured as a conical screw embodiment, with a distal point, and external thread, and a first and a second arm segment at the proximal end of the device.

Methods of reducing a fracture can include a closed or open reduction, as is known to those of ordinary skill in the art (e.g., such as can be found in Campbell's Operative Orthopaedics, S. Terry Canale, Editor; or Operative Techniques in Orthopaedic Surgery, Sam Wiesel, Editor in Chief; Hand Surgery, Editors: Richard Berger & Arnold-Peter Weiss, Lippincott, Williams & Wilkins, 2004; Rockwood and Green's Fractures in Adults; or any suitable online resource such as Orthopaedic Knowledge Online (OKO), etc.)

Following reduction of a fracture, methods can include verification of the position and alignment of the fracture fragments, e.g., using an imaging method, such as an x-ray or portable intra-operative mini-fluoroscopy, etc.

Methods of introducing the subject devices into a reduced fracture site can include the selection of the correct size and/or configuration of the subject device. Selection of an appropriate device can be performed by medical personnel, e.g., a surgeon, prior to a procedure or during the procedure, and can include evaluation of imaging studies (e.g., x-ray, CT, MRI), measurements taken of the fracture site, measurements obtained of the fracture site in the operating or procedure room, etc.

Methods of the subject invention can also include the use of any suitable tools for assisting in the use of the device. Such tools may include, e.g., forceps, tweezers, clamps, graspers, applicators, screwdrivers adapted for use with the device (e.g. a form fit shape such as a hex, cruciate, Torx, or Phillips design), guidewires, sheaths, catheters, and any specially-designed tools.

The device may be implanted through an incision providing access to the bone or fracture void. For example, for a distal radial fracture, the incision may be volar-radial, i.e., the Henry approach, or dorsal-radial between the first and second dorsal compartments. The fracture may be approached around the first dorsal compartment, releasing the distal-most fibers of the brachioradialis if necessary, and accommodating the instrumentation for distraction and reduction. This procedure may be performed with manual reduction. Although the exemplary embodiments disclosed herein refer to implanting the device within a distal radial fracture, it is understood that the device may be implanted in other bone fractures, voids, or defects of other bones, e.g., vertebral bodies, calcaneous, etc. as discussed above.

For implantation of the device within a distal radial bone or fracture void, the distal end of the device may be inserted first so that it becomes positioned on an ulnar side of the fracture, or the device may be inserted in any other orientation. In an embodiment in which the device includes a hinge which biases the first and second arm segments away from each other, the arms may be compressed together during implantation in order to decrease the insertion profile of the device. During implantation, the first arm segment may be oriented so that it is near the dorsal (larger) side of the fracture, and the second arm segment may be oriented so that it is near the volar (smaller) side of the fracture. After the device has been inserted into and properly located within the bone or fracture void, the compression on the arms may be released.

The device may also include bone securing elements and locking elements. After implantation, the bone securing elements may attach to bone adjacent the bone or fracture void, thereby securing the device within the bone or fracture void. For example, the bone securing element may attach to bone on a distal side of the bone or fracture void, and the bone securing element may attach to bone on a proximal side of the bone or fracture void, or vice versa. In addition, locking elements may engage with each other in order to constrain a maximum distance between the first arm segment and the second arm segment within the bone or fracture void.

In some instances, methods of treating a reduced fracture can include using the subject devices with additional elements, including fixation elements, or synthetic bone graft, or any suitable bone cement. For example, a fracture of the proximal tibia may be successfully reduced and treated with a device of the subject invention (e.g., a device with a conical screw configuration) however there may be an additional bone fragment or fragments that can be positioned with a fixation device, such as a K-wire, plates, or pin. In another example, a fracture of the proximal humerus may be successfully reduced and treated with a device of the subject invention (e.g., a device with a cylindrical coil configuration), however the fracture void may be large enough to require “filler” in the form of bone graft including autograft or allograft, synthetic graft, or bone cements (e.g., calcium phosphate, calcium sulfate, etc.). In yet another example, a compression fracture of a vertebral body may be successfully reduced and treated with a device of the subject invention (e.g., a pyramid-shaped device as in FIGS. 14B-D) however the fracture void may be large enough to require “filler” in the form of e.g., a calcium phosphate cement.

After the reduced bone fracture has been treated as described above, methods of treating the reduced fracture generally include a period of immobilization, e.g., with a splint or cast. In some instances, the period of immobilization can range from one week to 3 months, such as from two weeks to 2 months, or 1 month to 8 weeks. In embodiments in which an additional fixation device has been employed, the fixation device may need to be removed.

Methods of Manufacture

Methods of manufacturing the reduced bone fracture fixation device may include methods of forming the subject devices from a wire, include forming a resilient hinge by at least one loop, forming the first arm segment by at least one loop at one end of the hinge, and forming the second arm segment by at least one loop at the other end of the hinge. The reduced bone fracture fixation device may include a resilient hinge, a first arm segment attached to one end of the hinge, and a second arm segment attached to another end of the hinge, in which each of the hinge, the first arm segment, and the second arm segment includes at least one loop formed from wire.

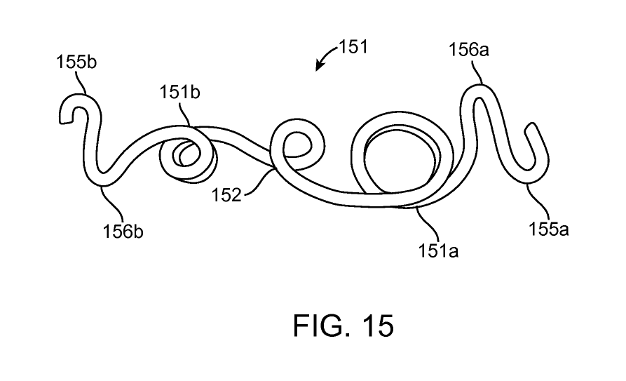

For example, FIG. 15 illustrates a partially formed embodiment of a reduced bone fracture fixation device. As shown, the device may be formed from a single wire that is bent and formed into each of the elements of the device. Alternatively, the device may be formed from multiple separate wires that are joined together.

As illustrated in FIG. 15, the hinge 152 is formed near the middle of the wire by at least one loop. The at least one loop of the hinge 152 may be a small single loop. The first arm segment 151a is formed on one end of the hinge 152, and the second arm segment 151b is formed on the other end of the hinge. The first arm segment 151a may include at least one loop, e.g., a large double loop. On an end of the first arm segment 151a opposite the hinge 152 there may be formed a first bone securing element 156a and a first locking element 155a. The second arm segment 151b may include at least one loop, e.g., a medium double loop. On an end of the second arm segment 151b opposite the hinge 152 there may be formed a second bone securing element 156b and a second locking element 155b.

The bone securing elements 156a and 156b may be formed as one or more loops, bumps, barbs, spurs, footholds, knuckles, coils, or other features to secure the device within a bone or fracture void. In addition, the locking elements 155a and 155b may be formed as hooks or other features to interlock or constrain the free proximal ends of the first and second arm segments 151a and 151b.

From the partially formed configuration shown in FIG. 15, the first and second arm segments 151a and 151b may be further formed to result substantially in the shape of the device shown in FIG. 5. That is, the first and second arm segments 151a and 151b may be bent such that a plane formed by the at least one loop of the first arm segment 151a may be substantially parallel to a plane formed by the at least one loop of the second arm segment 151b. In addition, the first and second arm segments 151a and 151b may be bent such that a plane defined by the at least one loop of the hinge 152 may be substantially perpendicular to each of the plane defined by the at least one loop of the first arm segment 151a and the plane defined by the at least one loop of the second arm segment 151b. As a result, the device may substantially form a trapezoidal, pyramidal, triangular, conical, oblong, ovoid shape, etc. Moreover, the device so formed may not require right- and left-handed configurations since the structure of the device may readily lend itself to any configuration and/or orientation.

In addition to bending or mechanical manipulation of wire, in some embodiments the reduced fracture fixation device can be formed by other techniques or methods, such as molding, stamping, machining, extrusion, scoring, or any other techniques or combination of techniques suitable for the particular material used. In some embodiments, the subject device can be formed from a planar element, as shown below.

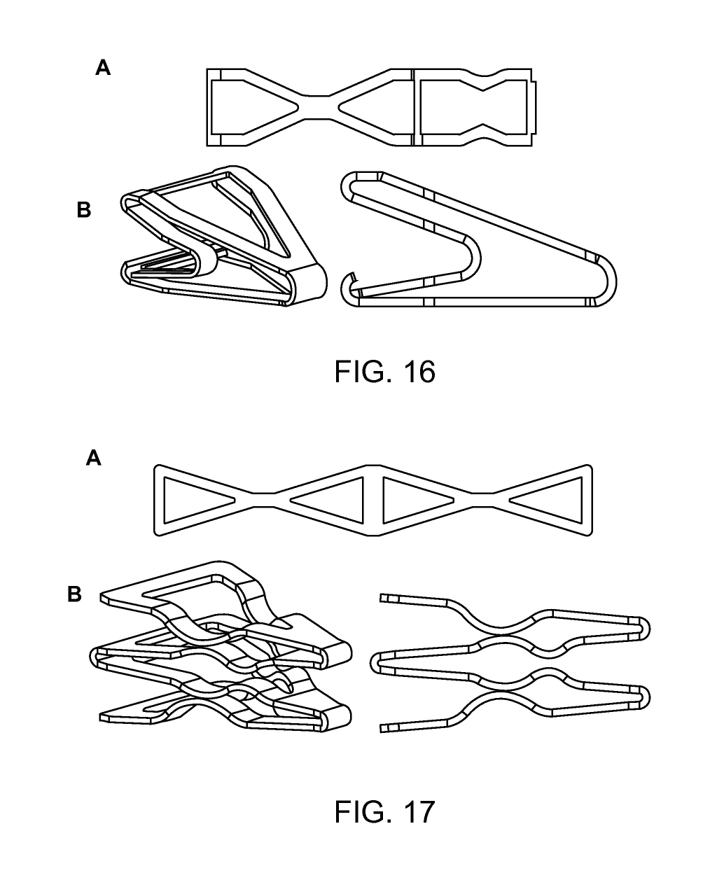

Methods of manufacturing the reduced bone fracture fixation device may also include methods of forming the subject devices from a planar element. FIG. 16 illustrates an embodiment in which a reduced bone fracture fixation device is formed from a single planar element, e.g., metal, which can be formed using any suitable method into the configuration as shown in FIG. 16A. The device can then be formed as shown in angled perspective view FIG. 16B and side view FIG. 16C. In some embodiments, the planar element as shown in FIG. 16A can be formed from more than one planar element joined together, using any methods suitable for the particular material used. In some embodiments, the final configuration of the subject device can include providing a self-closing fastener, e.g., similar to a safety pin closure, as shown in FIG. 16B, which can be integral to the device.

Another embodiment of the method of making the subject device is shown in FIG. 17, illustrating a reduced bone fracture fixation device formed from a single planar element of a different configuration. The final configuration of the device is as shown in angled perspective view FIG. 17B and side view FIG. 17C. As discussed above, the planar element as shown in FIG. 17A can be formed from more than one planar element joined together.

Methods of manufacturing the reduced bone fracture fixation device may include applying one or more coatings to all or a portion of the device, as discussed above. Such coatings include but are not limited to therapeutic agents, osteo-integration agents, hardening agents, anodizing treatments, etc.

Accordingly, the device may be formed so as to be implanted with minimal soft tissue invasion and bony disruption. Also, the device may be formed to restore metaphyseal and cortical collapse of a bone or fracture void. Further, the device may be formed to directly tension a bone or fracture void while maintaining reduction.

The description of the present invention is provided herein in certain instances with reference to a subject or patient. As used herein, the terms “subject” and “patient” refer to a living entity such as an animal. In certain embodiments, the animals are “mammals” or “mammalian,” where these terms are used broadly to describe organisms which are within the class mammalia, including the orders carnivore (e.g., dogs and cats), rodentia (e.g., mice, guinea pigs, and rats), lagomorpha (e.g., rabbits) and primates (e.g., humans, chimpanzees, and monkeys). In certain embodiments, the subjects, e.g., patients, are humans.

Kits

Also provided are kits that at least include the subject devices. The subject kits at least include a reduced bone fracture fixation device of the subject invention and instructions for how to use the device in a procedure.

In some embodiments, the kits can include a set of two or more reduced bone fracture fixation devices. In other embodiments, a set of devices can include at least three reduced bone fracture fixation devices, e.g., four or more, five or more, six or more, etc.

In some embodiments, a set of reduced bone fracture fixation devices includes two or more devices in which at least two of the bone fracture fixation devices are of different sizes. For example, in one embodiment a set of three reduced bone fracture fixation devices can be provided in a “small” size; a “medium” size; and a “large” size, which can vary in length along the longest axis of the device. The set of reduced bone fracture fixation devices can also be provided as a set of devices configured for a particular fracture site; e.g. proximal tibia, proximal humerus, hip, etc. In other embodiments, a set of devices e.g., for the distal radius, can be provided with both different sizes, and different configurations, in which some configurations might be more suited for a particular fracture site than other configurations.

In some instances, devices of different size, or devices for different sites may be labeled in any suitable manner to distinguish one size from another, or to distinguish a device for one site from another. For example, a “small” size wrist fracture fixation device can have an anodized treatment imparting a red color to the device, while the “medium” size fracture fixation device can have an anodized treatment imparting a blue color to the device.

In some instances, a kit can include one or more devices with one or more interchangeable elements, such that the interchangeable element that is best suited for a particular fracture site (e.g., optimal shape, size, degree of tension, etc.) can be selected and attached to the body of the device. The kit can also include a sizing and/or measuring tool, which can be disposable, for determining a desired size or configuration of bone fracture fixation device by measuring one or more distances, such as the distance between the surfaces of reduced fracture, the extent of the fracture, etc. The measuring tool can be any suitable measuring device, such as a sizer, a template, a caliper, a sterile disposable flexible tape measure, etc.

The kit can also include one or more tools configured to position a reduced bone fracture fixation device in a reduced bone fracture. The positioning tools can be disposable. Such tools may include, e.g., forceps, tweezers, clamps, graspers, applicators, screwdrivers adapted for use with the device (e.g. a form fit tip using for example a hex, cruciate, Torx, or Phillips design), guidewires, sheaths, catheters, and any specially-designed tools.

Other elements which can be included in the kit include any suitable bone cement (e.g., calcium phosphate cement), synthetic bone graft, BMP, PMMA (polymethylmethacrylate) cement, etc., or, which can be used with subject devices at the fracture site. The kits can also include one or more separate fixation elements, such as a K-wire, plates, etc.

The instructions for using the devices as discussed above are generally recorded on a suitable recording medium. For example, the instructions may be printed on a substrate, such as paper or plastic, etc. As such, the instructions may be present in the kits as a package insert, in the labeling of the container of the kit or components thereof (i.e. associated with the packaging or subpackaging) etc. In other embodiments, the instructions are present as an electronic storage data file present on a suitable computer readable storage medium, e.g., CD-ROM, diskette, etc. The instructions may take any form, including complete instructions for how to use the device or as a website address with which instructions posted on the internet may be accessed.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A reduced bone fracture fixation device, the device comprising:
a body dimensioned to be positioned in a reduced bone fracture, wherein the body comprises at least one tension element configured to exert a force on bone of the reduced bone fracture sufficient to maintain reduction of the reduced bone fracture.

2. The device according to claim 1, wherein the tension element comprises two arm segments.

3. The device according to claim 1, wherein the device further comprises a bone securing element.

4. The device according to claim 3, wherein the bone securing element comprises a thread.

5. The device according to claim 4, wherein the device is configured as a conical screw comprising a distal point, an external thread originating at the distal point and two proximal arm segments.

6. The device according to claim 5, wherein the external thread continues on the outer surfaces of the two proximal arm segments.

7. The device according to claim 6, wherein the two proximal arm segments are configured to apply opposing forces on bone of the reduced bone fracture.

8. The device according to claim 7, wherein the screw is hollow.

9. The device according to claim 1, wherein the device comprises two tension elements.

10. The device according to claim 2, wherein two arm segments are linked at their proximal ends by a looped wire segment.

11. The device according to claim 1, wherein the tension element comprises a conical coil.

12. The method according to claim 11, wherein the device further comprises a fastening element.

13. The device according to claim 1, wherein the tension element comprises a planar element.

14. The device according to claim 13, wherein the planar element comprises four arm segments.

15. The device according to claim 13, wherein the planar element comprises a triangular shape.

16. A method comprising:
reducing a bone fracture; and
introducing into the reduced bone fracture a device comprising a body dimensioned to be positioned in the reduced bone fracture, wherein the body comprises at least one tension element configured to exert a force on bone of the reduced bone fracture sufficient to maintain reduction of the reduced bone fracture.

17-26. (canceled)

27. A kit comprising:
a first reduced bone fracture fixation device comprising a body dimensioned to be positioned in a reduced bone fracture, wherein the body comprises at least one tension element configured to exert a force on bone of the reduced bone fracture sufficient to maintain reduction of the reduced bone fracture; and
at least one of:
a second reduced bone fracture fixation device;
a separate fixation element;
a tool configured to position the first reduced bone fracture fixation device in a reduced bone fracture;
a bone cement;
a bone graft; and
a sizing tool.

28. The kit according to claim 27, wherein the kit comprises a second reduced bone fracture fixation device.

29. The kit according to claim 27, wherein the kit comprises a separate fixation element.

30. The kit according to claim 27, wherein the kit comprises a tool configured to position the first reduced bone fracture fixation device in a reduced bone fracture.

31. The kit according to claim 27, wherein the kit comprises a bone cement.

32. The kit according to claim 27, wherein the kit comprises a bone graft.

33. The kit according to claim 27, wherein the kit comprises a sizing tool.
